(19) United States
(12) Patent Application Publication
(10) Pub. No.: US 2003/0235836 A1
(43) Pub. Date: Dec. 25, 2003

(54) Labeling of objects to be identified consisting of at least one DNA fragment

(76) Inventors: Ruben Antonio Simonetta, Cordoba (AR); Juan Carlos Jaime, Cordoba (AR); Julio Cesar Sabagh, Cordoba (AR)

Correspondence Address: BACHMAN & LAPOINTE, P.C., 900 CHAPEL STREET, SUITE 1201, NEW HAVEN, CT, 06510, US

(21) Appl. No.: 10/307,012

(22) Filed: Nov. 27, 2002

(30) Foreign Application Priority Data
Jun. 20, 2002 (AR) 02102319

(51) Int. Cl. C12Q 1/68; C07H 21/04

(52) U.S. Cl. 435/6; 536/24.3

(57) ABSTRACT

A consistent marker is provided which contains at least one fragment of DNA, preferably a plurality of polymorphic DNA fragments of the type microsatellites (STR) and single nucleotide polymorphisms (SNP) microencapsulated and bound to a selected system of detection such as magnetic microspheres; pigments and a fluid with electrical properties, and/or fluorescent to ultraviolet and/or infrared radiation.

### BACKGROUND OF THE INVENTION

(1) Field of the Invention

The present invention relates to a marker of objects to be identified which marker includes at least one fragment of DNA. The present invention also relates to procedure for the incorporation of the marker in the objects to be identified and a method for the identification of the marked objects.

(2) Prior Art

Deoxyribonucleic acid (DNA) is a molecule of great size in which the genetic information of an individual is stored.

Some sequences have specific functions, for example the genes and their regulators. Others, seemingly silent, are still ignored. They are extremely abundant inside the genoma.

Some sequences vary from one individual to another. For that reason, the comparison of these polymorphic regions constitutes the basis of molecular identification.

The polymorphic regions are inherited from parents to children (mendelian inheritance). They allow the identification of a person by means of the comparison with individuals potentially related biologically.

The development of the techniques of DNA Typing that allow the identification of any individual, has already entered in its adolescence.

Fifteen years have passed since this discovery that allowed one to identify with great accuracy a person through the study of his DNA and the comparison with the DNA of his relatives. Although the underlying conceptual aspects to the process of identification have not varied in a considerable way, the methodological aspects have experienced a vertiginous evolution.

The detection of new markers, the automation of some experimental stages, as well as the creation of computerized systems with great storage capacity and data analysis, has been the tendency in the field of the human identification.

From the year 1980, polymorphic sites are known in the human genoma, being the first ones in being detected, those located in near regions to some genes like that of insulin.

However, the detection of multiple variable regions (MLP) have made possible the development of the systems of individual identification that are not restricted to the human species, but also includes all animal species and vegetables.

This great contribution to scientific knowledge, with unsuspected application uses, was carried out by the English scientist Alec Jeffreys and his collaborators in the year 1985.

Such variable regions that are dispersed in the whole genoma, allowed to show specific characteristics that are inherited in mendelian way.

The impossibility of locating in exact form the position of these regions in the genoma only allows the obtaining of a “molecular phenotype” and such phenotypic comparison makes possible the personal identification.

Although this type of comparison is objective, its statistical evaluation based on the populational analysis and its use allowed the development of the first molecular system of human identification of great social impact that was intensely used until the decade of the '90s.

By means of the use of probes, it was able to generate multilocus patterns to rake in human libraries (the whole genetic information of an individual contained in microbial hosts). It made possible the detection of variable sites in the genoma, arising in this way the markers of specific locus (SLP) able to define the genotype of an individual for a certain locus.

This method allowed the replacement of its predecessor and it contributed in great measure to the standardization of the analytic procedures of human identification, very particularly those faced by the FBI in the United States and by the Forensic Science Services of England.

These robust genetic markers constituted the identification tool until the end of 1995.

Later on, the use of microsatellites or short tandem repeats (STR) that consist of polymorphic sequences of short repetition units and whose analysis depends on amplification with the Polymerase Chain Reaction (PCR), were replacements.

These genetic markers are broadly utilized in the forensic laboratories all over the world for the identification of people, so much to establish relationships, like to identify the presence of individuals in a certain scenario through the findings of biological samples.

These markers exhibit characteristics that make them appropriate for forensic analysis, among others the high sensibility, because it requires minimum quantities of DNA for the analysis.

It is due to this sensibility, to the speed of the analysis, and to the simple interpretation of the results, that the microsatellites or STRs became the favorite forensic markers.

Although at the moment the number of STRs is very high, a group of validated markers have been selected because they have been incorporated into standardized systems of analysis in commercial kits.

This group or kit of markers allows one to analyze in simultaneous reaction up to 16 polymorphic human markers, thirteen of which constitute the basic group that has allowed the design of “intelligent databases.”

The inventors know that the Single Nucleotide Polymorphisms (SNP) appear in every one of each thousand of bases pairs, totaling in consequence, three millions in the human genoma.

Patent document number CN1302905 refers to anticounterfeiting material containing metallic ions of DNA prepared by mixing an aqueous solution of a soluble metallic salt with high coordination power with a solution of DNA and alcohol to obtain a decanted solution of soluble water M-DNA with gelatin, dextrin, the aqueous solution of starch soluble or rubber for the marked or ink to impress.

Patent document number CN1306266 refers to a card of genetic identification, a method for the preparation of the card including obtaining the owner's DNA, the prosecution of the information, and its impression.

German document number DE4446042 refers to a card that understands an entity that provides a plurality of identifications of different species related chemically, for example by means of the use of a transporter of a material such as polystyrene, nitrocellulose, protein, polysaccharide or alcohol. The identifications can be enzymes, antibodies, antigens, and DNA. The cards can be used to authenticate bank notices, perfumes, documents, etc.

U.S. Pat. No. 6,167,518 refers to digital certificates formed by the digital representation of a biological characteristic of the registrant; for example the DNA chromosomal of the registrant. The representation has a personal message that is transmitted to the certificate itself. The identity of the registrant is verified in a remote way. The characteristics can be extracted from the certificate and compared.

U.S. Pat. No. 6,213,391 refers to an identification system generated by starting from a biometric characteristic distinctive (for example: voice analysis, DNA, etc.). The biometric information is used in a variety of functions such as the control and security of transactions. An algorithm is provided for the creation of a key number for its use like a secondary identification code.

U.S. Pat. No. 6,256,737 relates to a system, method, and software that uses biometric measures for the authentification of users resources. A biometric control determines the method in which the user can be authenticated by the system. The execution includes the use of at least one biomedical parameter. It uses a scientific method for the user for comparison with an unique characteristic, such as for example the DNA. The method is disclosed to store the identification parameters.

U.S. Pat. No. 6,312,911 relates to a method to hide a message coded in a microdot using DNA and a method for the use of a mark with a coded message to identify objects.

Patent document number WO0068431 relates to a stenographic method to hide messages coded in DNA. The method understands the uses of a hidden sample of DNA in a microdot and the labeling and authentification of objects of interest.

Patent document number WO0165375 relates to a system, method, and software that uses a measurement to authenticate users. It uses unique personal characteristics obtained by biometrics forms and compared with those stored in a memory. The unique characteristics are, among others, the geometry of fingers and hands; analysis of facial and retinal image, voice, DNA, etc.

The search of elements, dispositions, and mechanisms that grant greater security to transactions that habitually are carried out are needed.

From the remote days of the creation of money until now, man has looked for ways to avoid robberies, deceits and falsifications perfecting the impression methods and incorporating safe-deposit elements that the technical advances and the incipient quality of the equipment offered to the greater public have been overcoming.

To the erased values of checks to redo them for superiors values, it has been opposed the marking of the paper.

The falsification of paper currency and documentation in general have taken to the adoption of papers and special inks, optical inks, incorporation of safe-deposit elements, protection with translucent films, etc.

The inventors know the existence of elements that are unique for each person. Such is the case of fingerprints and such is also, the case of DNA.

Indeed, each individual possesses a particular biological signal and the current techniques allow the identification of it with practically, absolute certainty.

Although this is the way, it does not escape the knowledge of the inventors that the incorporation of DNA coming from a live person to an object to achieve a positive identification would be easily utilized by forgers, because it would be enough to be near the possessor of such DNA to obtain a sample from him.

Indeed, hair, saliva deposited on a glass, a drop of blood, and epithelial cells would be enough to obtain the necessary DNA to be added to the false object.

### SUMMARY OF THE INVENTION

Keeping in mind the enunciated possibility, the inventors have considered that an object to be identified will be marked with at least one fragment of DNA, and still better, with a combination of fragments of DNA. Although a forger may be able to obtain the necessary DNA to proceed to the incorporation to the object, he will be ignorant as to which of such fragments, between the existent millions or their possible combinations, he will have to use to achieve his objective.

It is for that reason that it is an objective of the present invention to count with a marker of objects to be identified that consists of at least one fragment of DNA.

It is also an objective of the present invention to count with a procedure that allows the incorporation of this marker into the object or objects to be identified.

It is also another objective of the present invention to count with a method that allows the identification of the marked objects with the marker.

In order to achieve a labeling of objects that cannot be reproduced by forgers, the inventors proceed to incorporate in such objects polymorphic fragments of DNA, such as microsatellites (STR) and Single Nucleotide Polymorphisms (SNP) since these appear in all live beings, so much of animal origin as of vegetable origin.

In the present application, the inventors disclose a procedure also to incorporate these fragments to the objects to be identified and a method that allows the identification of the marked objects.

### DETAILED DESCRIPTION OF THE PREFERRED EMBODIMENT(S)

With the purpose of making comprehensible the present invention so it could be taken to the practice with ease, the following paragraphs will provide a description of the preferred way of realization, with the objective being purely demonstrative but not limitative of the invention whose components will be able to be selected among diverse equivalents without moving itself away from the principles of the invention described herein.

The present invention relates to a marker of objects to be identified that discloses the incorporation of at least one fragment of DNA and, specifically, the incorporation of polymorphic fragments of DNA of the type of microsatellites (STR) and single nucleotide polymorphisms (SNP) to the objects to be identified.

The invention also relates to a procedure that includes a first step of a live being's selection to proceed to the extraction of DNA from any one of its cells; a second step of purification of the obtained DNA; a third step of amplification of the polymorphic fragments of the type of microsatellites and single nucleotide polymorphisms; a fourth step of concentration and/or microencapsulation of the DNA; a fifth step of solubilization of the microcapsules of DNA; a sixth step of determination and/or correction of the degree of fluidity and concentration of the solution; and a seventh step of incorporation of the solution in an appropriate applicator and labeling of the wanted object.

The present invention also relates to a method for the identification of the marked object. In a first step, an appropriate detecting technique is used to detect an incorporated component to the solution and to individualize the marked object. In a second step, the key of the incorporated marker is obtained. In a third step, the authentification of the marked object by means of the realization of the necessary analysis is obtained.

Once the components of the invention are established, as well as the sequence of developed stages to explain its nature, they are supplemented subsequently with the functional and operative relation of the provided results.

In order to having a marker of objects to be identified that constitutes a secure way to protect valued items, the present invention proposes the incorporation of a marker to these objects.

Preferably, the inventors have considered that this marker will be a chemical compound that can be detected with posteriority.

The inventors prefer the use of deoxyribonucleic acid (DNA) as the marker of the objects to be identified.

Particularizing, the inventors have considered that in attention to the great variety of combinations, it is rather impossible to reproduce the labeling performed by means of the incorporation of polymorphic fragments of DNA of the type of microsatellites and single nucleotide polymorphism to the objects to be identified.

It is convenient to clarify that when mention is made to DNA, the reference is in particular to the mentioned polymorphic segments. That is to say, those which make all live beings of the Earth to be different.

Also, when mention is made to individual polymorphic segments, it should be understood that such mention is only related to Short Tandem Repeats/Microsatellites (STR) and Single Nucleotide Polymorphism (SNP).

It is also necessary at this point, to make the exception that the univitelian twins will only present polymorphic segments in common, so that would not allow their differentiation.

However, in the case of univitelian twins, it will be able to obtain a differentiating labeling based on the combination of the individual polymorphic segments that are selected.

Indeed, when using a certain combination, the only person that knows the exact site inside the thousands of polymorphic sites of the live being's genoma that has been used in the labeling of the object is the owner of it.

If necessary, and once this site is revealed, it will enable a comparative analysis to be carried out in any laboratory of the world that uses DNA Typing for people identification purposes.

This circumstance is of supreme utility mainly in those cases where it is necessary to obtain an identification of the object in other jurisdictions, and mainly, when it should be appealed to justice.

In relation with the procedure for the incorporation of a consistent marker including at least one fragment of DNA in the object to be identified, the present invention relates to a plurality of steps where in a first step, you select a live being and proceed to extract the DNA that will be used.

The use of the marker with at least one fragment of a live being's DNA is proposed by the inventors. It should be interpreted in a wide sense, that is to say that whoever takes the decision of carrying out the labeling of their objects, will be able to select itself as the donor of the DNA fragments or will be able to select any live being, human being, animal or vegetable. As a consequence, the tiny possibility of reproduction of the marker by forgers decreases even more.

The extraction of the DNA is carried out starting from cells or corporal fluids obtained by regular techniques, such as buccal swab; blood punction; the collection of epithelial cells, hair follicles, and the like.

In a second step, the obtained DNA is liberated in a solution compound by “Tris-ClH 10 mM”-EDTA 0.1 (mM), SDS to 20% (weight/volume) and Proteinase K 10 mg/ml. to proceed then to the purification with Phenol/Chloroform—10/9 (volume/volume).

In a third step, STRs and/or SNPs are amplified by using the Polymerase Chain Reaction (PCR) set forth in U.S. Pat. Nos. 4,683,195; 4,683,202; and 4,800,159, which are incorporated by reference herein.

The mixes that are placed into a thermocycler, contain the sample of DNA in a concentration of between 6 pgr. and 0.05 microgr., a PCR Buffer solution 10×, dNTP 10×, primers that flank the polymorphic region 10× of each one and Taq polymerase of 5000 units per ml.

In a fourth step, and in order to preserve the DNA from degradation, it is proceeded to concentrate by ultracentrifugation for which microconcentrators such as Centricon 100 are used; and microencapsulating with the technique of phase inversion.

In this step, the polymorphic DNA to be microencapsulated is dissolved in a solvent, and then, in the same solvent, a polymer is dissolved to a final concentration of between 0.25% and 10% weight/volume.

The polymer used can be selected indistinctly between those biodegradable or those non-biodegradable.

The preferred biodegradable ones are those such as lactic and glycolic acids and esters such as polyanhydrides, polyurethanes, butyric polyacid, valeric polyacid, and the like.

On the other hand, inside the non biodegradable polymers the preferred ones are vinyletylene acetate and acrylic polyacid, but also acceptable are the use of polyamides and copolymers, as a mixture thereof.

The utilized polymers can also be selected from natural ones, such as dextran, cellulose, collagen, albumin, casein, and the like.

The resulting mixtures are introduced later on in a non-solvent in a relation solvent/non-solvent of at least 1/40 up to 4/200 to obtain the spontaneous formation of microcapsules.

In this step, the organic solvent is selected from chloroform and methylene chloride, and the preferred non-solvents are ethanol and hexane.

Alternatively, in the fourth step, microcapsules take place with polycationics agents such as poly-L-lysine and ClNa, in a first stage a selected polymer is dissolved among those enumerated in an organic solvent such as chloroform.

In a second stage of the fourth step, the polymorphic DNA is dissolved in water producing a first aqueous phase.

In a third stage of the fourth step, the organic phase is emulsified with the first aqueous phase to obtain a first milky emulsion.

In a fourth stage of the fourth step, the ClNa is dissolved in alcohol polyvinylic, producing a second aqueous phase.

In a fifth stage of the fourth step, the first milky emulsion is emulsified with the second aqueous phase to produce a second milky emulsion.

Lastly, in a sixth stage of the fourth step, the organic solvent of the second milky emulsion is evaporated, taking place the formation of microcapsules containing polymorphic DNA.

Alternatively, in the fourth step, the DNA can be bound to magnetic microspheres or to pigments that are visible or not to the human eye or to pigments with electrical properties and/or fluorescent to the ultraviolet and/or infrared radiation.

Additionally, to mask the DNA fragment or the DNA fragments selected and to make even more difficult the falsification of the labeling, one can use a combination of the techniques described in the fourth step; as well as the incorporation of other fragments of DNA different to the chosen ones.

In a fifth step, it is proceeded to solubilize the microspheres of DNA or the DNA microencapsulated in a solution containing sensitive substances to an ultraviolet radiation, such as fluorescein for example, tetrametil rhodamine, rhodamine 3, texas red, and the like, and/or sensitive substances to infrared radiation such as upconverted phosphor like gallium oxysulfur or lanthanides ions bound to a naphthalene group, and the like.

So much the sensitive substances to the ultraviolet radiation as to the infrared radiation can be added free or microencapsulated with anyone of the techniques previously described.

In an alternative embodiment, it is proposed to solubilize the microspheres of DNA or the microencapsulated DNA in a mixture of substances that are sensitive to the infrared radiation and the ultraviolet, for example containing between 0.0001 and 0.02% ftalocianine in weight with a wavelength that oscillates between 670 and 720 nm and between 0.05 and 0.5% in weight of a selected fluorosphor among stilbene, dihydropyrazole, coumarin, carbostirilo and a compound of pirene with a wavelength that oscillates between 250 and 380 nm.

In a sixth step, it is proceeded to determine and if necessary to correct the degree of fluidity and concentration of the solution that should be appropriate to facilitate its application in the objects to be marked.

It has been considered that the degree of fluidity should allow that the applicator deposits a solution with a concentration of between 6 pgr and 10 microgr. of marker per mm² surface.

In a seventh step, the marker is incorporated to an applicator that can be selected among a pen (pen having a nip, a ballpoint, or a felt tip), diverse types of filters, drawing instruments, paintbrush, stamp or some automatic machine such as inkjet printer and the like.

In an alternative embodiment an intermediary is used among the solution containing the marker and the object to be marked, in which the intermediary is absorbed into the solution.

The intermediary can be selected from among diverse substances, such as nitrocellulose, paper, wood, cardboard, plastic material, charged nylon, cloth, organic substances in form of drops or gel, inorganics, and the like.

Lastly, with the selected applicator, it is proceeded to label the designated objects.

The method for the identification of the marked objects with at least one fragment of DNA includes a step of detection of the object by means of an appropriate system.

To do so, the system will be able to consist of a filter that facilitates the visualization of the pigments or the incorporated elements to the marker or a detector that allows one to verify the presence of some wavelength radiation with the sensitive substances incorporated, or the magnetic particles or the characteristics of conductivity of the solution, and the like.

Diverse types of magnetic detection as well as the electronic system verification to detect a certain component according to their grade of conductivity are known in the prior art.

Detected in a first step is the object to be identified. In a second step, it is proceeded to obtain the key of the incorporated marker. In a third step, it is proceeded to the authentification of the marked object proceeding to carry out the necessary analysis for the typing of the polymorphic fragments of DNA by using the Polymerase Chain Reaction.

The third step will only be able to be carried out when the owner of the marked object reveals which are the oligonucleotides segments that has been placed on the object. Starting from the supply of this fact, any forensic laboratory in the world that uses the technique of Polymerase Chain Reaction, as described, will be able to proceed to amplify and detect the polymorphic fragments of STR/SNP.

The detection of the fragments will be able to be carried out by means of procedures and techniques that are usually used in the previous art such as gels according to J. M. Robertson (1994); capillary electrophoresis according to McCord (1993); detection for multiple hibridization or multiple capillarity proposed by Y. Wang (1995), by the use of the microchips like Woolley (1996); by Mass spectrometry according to Becker (1997); etc.

In turn, it will be able to detect the single nucleotide polymorphism by means of a conformational analysis of unique chain as demonstrated by Orita and others (1989); allelic oligonucleotide specific as Landeegren and others indicated (1988); multiple extension of primers according to Syvanen and others (1990) or other technologies among those mentioned as chips, mass spectrometry, etc.

This way, in the event of a controversy, the rights of all the parties are guaranteed since it is possible to reproduce the concerning test to the identity of the marked object whenever it is necessary and in any place in the world since, like it was said, the markers STR and SNP are those that are routinely used for the identification of people.

On the other hand, it should be understood that these markers are recommended by the International Society for Forensic Genetics.

With the marker disclosed herein it will be able to identify with absolute certainty countless objects such as paintings, sculptures, inputs of sport, works of art, handy crafts, videocassettes, recorders, televisions, home objects, computers, printers, software, office elements and business equipment.

Also, it will be able to identify perfumes, clothes, wallets, briefcases, boxes, parts of automobiles, airplanes, bicycles, paper money, paper currency, checks, notarial documents, identification cards, driver's licenses, passports, visas, credit cards, telephone cards, and such similar objects as academic certificates, inventories, lottery tickets and other games of chance.

In this way one of the possible sequences of stages has been pointed out, to sum up the invention and the way it works, and the documentation is supplemented with the synthesis of the invention contained in the clauses that are next added.

What is claimed is:

1. A marker of objects to be identified that comprises at least one fragment of DNA bound to at least one system of detection selected from the group consisting of magnetic microspheres, pigments, a fluid with electrical properties, fluorescent to ultraviolet radiation and fluorescent to infrared radiation.

2. Marker according to claim 1, wherein said at least one fragment of DNA is a plurality of polymorphic fragments of DNA of the type of microsatellites (STR).

3. Marker according to claim 1, wherein said at least one fragment of DNA is a single nucleotide polymorphism (SNP).

4. Marker according to claim 1, wherein said at least one fragment of DNA is microencapsulated.

5. Marker according to claim 1, wherein said at least one fragment of DNA is microencapsulated and is incorporated in a solution containing at least one substance sensitive to ultraviolet radiation.

6. Marker according to claim 1, wherein the at least one substance sensitive to ultraviolet radiation is selected from the group consisting of fluorescein, tetrametil, rhodamina, rhodamina 3 and texas red, and mixtures thereof.

7. Marker according to claim 1, wherein said at least one fragment of DNA is microencapsulated and is incorporated in a solution containing at least one substance sensitive to infrared radiation.

8. Marker according to claim 7, wherein said at least one substance is an upconverting phosphor as lanthanide ions bound to a naphthalene group.

9. Marker according to claim 1, wherein said at least one fragment of DNA is microencapsulated and is incorporated in a solution containing substances or pigments that are sensitive to ultraviolet radiation and/or infrared radiation.

10. Marker according to claim 1, wherein said at least one fragment of DNA is microencapsulated and is incorporated in a solution formed by a mixture of substances sensitive to infrared radiation and ultraviolet radiation.

11. Marker according to claim 10, wherein said solution contains between 0.0001 and 0.02% ftalocianine in weight with a wavelength of between 670 and 720 nm and between 0.05 and 0.5% in weight of a fluorosphor selected from the group consisting of stilbene, dihydropyrazole, coumarin, carbostirilo and compound of pirene and said fluorosphor has a wavelength of between 250 and 380 nm.

12. Procedure for incorporating a marker in an object to be identified comprising: extracting a sample containing DNA from a selected live being; determining and optionally correcting a degree of fluidity and concentration of a solution containing the extracted DNA and incorporating the solution in an applicator; and labeling the object to be identified.

13. A procedure according to claim 12, further comprising liberating the DNA in a solution compound with Tris-ClH 10 mM-EDTA 0.1 mM, SDS to 20% (weight/volume) and Proteinase K 10 mg/ml., purifying the DNA with Phenol/Chloroform 10/9 (volume/volume); performing a step of microsatellites and single nucleotide polymorphism amplification using polymerase chain reaction, obtaining a concentration of among 6 pgr. and 0.05 microgr. of the DNA sample, a solution PCR Buffer 10×, dNTP 10×, primers that flank the polymorphic region 10× of each one and Taq polimerase of 5000 units per ml., and placing the solution and the DNA into a thermocycler; concentrating the DNA by ultracentrifugation; dissolving the solution containing DNA in a solvent and a polymer in a concentration of between 0.25 and 10% weight/volume, introducing a resulting mix in a non-solvent in a relation solvent/non-solvent of between 1/40 up to 4/200 and microencapsulating the DNA, and solubilizing the microcapsules of DNA in a solution containing substances sensitive to ultraviolet radiation and/or infrared radiation.

14. A procedure according to claim 13, further comprising producing the microcapsules containing polications agents selected from the group consisting of the poly-L-Lysine and ClNa, by dissolving a polymer in an organic solvent, dissolving a polymorphic DNA in water producing a first aqueous phase; emulsifying the organic solvent with the first aqueous phase to obtain a first milky emulsion; dissolving the policationic agents in alcohol polyvinylic and water producing a second aqueous phase; emulsifying the first milky emulsion with the second aqueous phase to form a second milky emulsion, and evaporating the organic solvent of the second milky emulsion and producing microspheres containing polymorphic DNA.

15. A procedure according to claim 13, wherein the utilized polymer is biodegradable and is selected from the group consisting of lactic acid, glycolic acid, and an ester.

16. A procedure according to claim 15, wherein the ester is selected from the group consisting of polyanhydrides, polyurethanes, butiric polyacid and valeric polyacid, and mixtures thereof.

17. A procedure according to claim 13, wherein the utilized polymer is non-biodegradable and is selected from the group consisting of vinyletilene acetate, acrylic polyacid, a polyamide, a copolymer, and mixtures thereof.

18. A procedure according to claim 13, wherein the utilized polymer is a natural polymer selected from the group consisting of dextran, cellulose, collagen, albumin, and casein.

19. A procedure according to claim 13, wherein the DNA is bound to magnetic microspheres or pigments that are visible or invisible to the human eye.

20. A procedure according to claim 13, wherein the DNA is bound to a fluid with electrical properties and/or fluorescent to ultraviolet and/or infrared radiation.

21. A procedure according to claim 14, wherein the organic solvent is selected from the group consisting of chloroform and methylene chloride, and the non-solvents are selected from the group consisting of ethanol and hexane.

22. A procedure according to claim 13, further comprising adding a mix of substances sensitive to infrared radiation and ultraviolet radiation containing between 0.0001 and 0.02% in weight ftalocianine with a wavelength of between 670 and 720 nm and between 0.05 and 0.5% in weight of a fluorosphor selected from the group consisting of stilbene, dihydropyrazole, coumarin, carbostirilo and a compound of pirene with said selected fluorosphor having a wavelength of among 250 and 380 nm.

23. A procedure according to claim 13, wherein said concentrating step comprises adding microconcentrators of the type of the Centricon 100.

24. A procedure according to claim 13, wherein said determination step comprises providing a solution having a concentration of between 6 pgr and 10 microgr of marker per mm² of surface.

25. A procedure according to claim 13, wherein said incorporating step includes selecting the applicator from the group consisting of a pen having a nip, a ballpoint pen, a felt tip pen, a filter, a drawing instrument, a paintbrush, a stamp and automatic machines.

26. A procedure according to claim 13, wherein said incorporating step comprises incorporating into the solution containing the marker an intermediary selected from the group consisting of an organic substance in drop form, an organic substance in gel form, and an inorganic substance.

27. A procedure according to claim 13, wherein said incorporating step comprises incorporating an intermediary selected from the group consisting of nitrocellulose, paper, wood, cardboard, plastic material, charged nylon and cloth.

28. Method for identifying an object marked with at least one fragment of DNA comprising detecting of an object to be identified, obtaining identification of the at least one DNA fragment in the marked object and authenticating the marked object by detecting primers that flank a polymorphic region using Polymerase chain reaction.

29. Method according to claim 28, wherein the detecting step is carried out by means of a filter for visualizing at least one of pigments and elements incorporated into the marker.

30. Method according to claim 28, wherein the detecting step is carried out by detecting at least one of a radiation type such as the wavelength, magnetism, and/or conductivity with at least one radiation sensitive substance, magnetic particles, and solution conductivity characteristics.
